(19) United States
(12) Patent Application Publication
(10) Pub. No.: US 2021/0407628 A1
(43) Pub. Date: Dec. 30, 2021

(54) METHOD FOR PERFORMANCE PREDICTION OF GLASS SYSTEM

(71) Applicant: SOUTH CHINA UNIVERSITY OF TECHNOLOGY, Guangdong (CN)

(72) Inventors: Zhongmin YANG, Guangdong (CN); Guoquan QIAN, Guangdong (CN)

(73) Assignee: SOUTH CHINA UNIVERSITY OF TECHNOLOGY, Guangdong (CN)

(21) Appl. No.: 16/764,406

(22) PCT Filed: Jul. 25, 2019
(86) PCT No.: PCT/CN2019/097785

§ 371 Date: May 15, 2020

(30) Foreign Application Priority Data
Jun. 27, 2019 (CN) 201910566855.3

(51) Int. Cl. G16C 60/00; C03C 3/253; C03C 3/16; C03C 3/12; C03C 3/04; G16C 20/30; G06F 30/25

(52) U.S. Cl. CPC G16C 60/00; C03C 3/253; C03C 3/16; G06F 30/25; C03C 3/04; G16C 20/30; C03C 3/122

(57) ABSTRACT

A method for performance prediction of a functional glass system, which includes the following steps: determining species of atoms for structural search according to components of a target glass system; performing structural search based on a first principle to search out compounds that can be formed by interaction between the atoms; comparing a formation energy and a phonon spectrum of each of the compounds to obtain stable compounds; constructing a glass structural composition diagram according to the stable compounds, microstructural units of a glassy compound adjacent to a target glass composition point are structural genes of the glass; and calculating a property of the target glass according to a leverage model formula of a multiplex glass system, the leverage model formula of the multiplex glass system being P0=Σᵢ₌₁ⁿPi×Li.

### TECHNICAL FIELD

The present invention relates to the field of glass material research, and more particularly, to a method for performance prediction of a glass system.

### BACKGROUND

Functional glass materials have become an essential part of modern life and are widely used in various fields including daily life, national defense construction, biological medicine, safety protection, laser weapon, laser medical treatment, etc., and play an important role in promoting development of social civilization. With the flourish of the above fields, higher requirements are put forward on research and development speeds, research and development costs and performances of the functional glass. Therefore, it is especially important to achieve efficient and low-cost research and development of the functional glass materials. However, although glass has been used for thousands of years, an essential problem on the relationship among composition, structure and performance of the glass is not fully understood yet, and the glass is still a mysterious material.

According to traditional research modes, a glass researcher roughly gives a glass formula meeting conditions firstly according to literature information and data accumulated in his or her laboratory, then fuses glass components at a high temperature to prepare the glass, tests corresponding performances of the glass, adjusts the formula according to test results, and tests repeatedly in a sequential iteration until a glass product meeting specific conditions is finally obtained. Although such mode of trial-and-error method has made multiple important achievements in the research and development of the functional glass materials, the mode has problems of insufficient understanding on a glass system structure, a long test period, high costs and a low efficiency, which seriously restrict efficient and rapid research and development of the functional glass.

The United States proposed “Materials Genome Initiative” in 2011 first, aiming at revitalizing American industry and accelerating the research and development of new materials. Subsequently, China carried out corresponding layouts actively. The material genome method is a significant leap in the research and development of the materials, and is a “propeller” for the research and development of new materials. A sequential iteration method in a traditional trial-and-error method is replaced by a high-throughput concurrent iteration method in the material genome method, and the “experience-guided experiment” is gradually changed to the “combination of theoretical prediction and test verification” in a research and development mode of the materials, so as to realize a target of “shortening a research and development cycle by half and reducing research and development costs by half” and accelerate a process of “discovery-development-production-application” of the new materials. A research concept of the material genome method is rich in contents, including modeling research, high-throughput test characterization technique, computer simulation, data mining, machine learning, artificial intelligence, etc. The proposal of the material genome method provides a brand-new research concept for the research and development of the materials, and is an innovation to the traditional trial-and-error method of the materials. However, at present, the material genome method is mainly used in fields of semiconductor materials and metal materials with relatively simple components and structures, and the material genome method is rarely used in the glass materials because of the disorder and diversity of the glass material structure.

Based on the previous researches, we have found that the structure of glass is very similar to the glassy compound in the corresponding phase diagram, and the performances of the glass can be predicted by the neighboring glassy compound. On this basis, we propose that the glassy compounds in the corresponding phase diagram of the glass are the structural genes of the glass. For a glass system with relatively complete phase diagram data, we provide a method for predicting a luminescent performance of laser glass from the perspective of a glass phase diagram, find the structural genes of the glass system by consulting a large number of literatures and searching the phase diagram database, and then predict the luminescent property of target glass by using the property of the glass genes. However, this technology has two major problems as follows. (1) Important data in the literature and the phase diagram database often undergoes the problems of inconsistent test conditions and error in a test method, and the problems are very unfavorable for judging and finding the structural genes of the glass. (2) The method is invalid for a glass system with a relatively incomplete phase diagram database. (3) The predicted performance provided by the method is single, and the predicted glass variety is single. Therefore, how to accurately and quickly predict the performance of the glass system without the phase diagram data has become a key technical problem in the field.

### SUMMARY OF THE INVENTION

On this basis, it is necessary to provide a method for performance prediction of a glass system.

The present invention provides a method for performance prediction of a multiplex glass system, which includes the following steps:

determining species of atoms for structure searching according to components of the multiplex glass system;

performing structural search based on the first principle to search out compounds that can be formed by interaction between the atoms;

comparing a formation energy and a phonon spectrum of each of the compounds to obtain stable compounds;

constructing a glass structural composition diagram according to the stable compounds, microstructural units of a glassy compound adjacent to a composition point of a target glass are structural genes of the glass; and

calculating a property of the target glass according to a leverage model formula of the multiplex glass system which is P₀=Σᵢ₌₁ⁿPi×Li, wherein the multiplex glass system includes n components, P₀ is the property of the target glass, Pi is a property of the structural gene of the target glass, and Li is a content of the structural gene of the target glass.

The present invention provides a method for performance prediction of a binary glass system, which includes the following steps:

performing structural search based on the first principle to search out compounds that can be formed between every two atoms or among every three atoms in components of a target glass, and obtaining formation energies and phonon spectrums of the compounds by calculating;

comparing the formation energies and the phonon spectrums of the compounds respectively to obtain stable compounds;

drawing a composition triangle by taking composition atoms of the target glass as vertexes, and marking coordinates of the stable compounds in the composition triangle to obtain a binary glass system composition diagram;

finding out a composition coordinate of the target glass in the binary glass system composition diagram, microstructural units of glassy compounds corresponding to two stable compounds adjacent to the composition coordinate are structural genes of the target glass; and

calculating a property of the target glass according to a leverage model formula of the binary glass system which is P₀=P1×L1+P2×L2, wherein P₀ is the property of the target glass, P1 and P2 are properties of the structural genes of the target glass, and L1 and L2 are a content of the structural genes of the target glass.

In one of the embodiments, the property is at least one of a mechanical property, a magnetic property, an electrical property, a luminescent property and a thermal property.

In one of the embodiments, the property is at least one of a density, a refractive index, a fluorescence full width at half maximum, an effective line width, a absorption cross-section and a peak emission cross-section.

In one of the embodiments, performing the structural search based on the first principle calculation method is to perform high-throughput structural search calculation using a first principle structural search software.

In one of the embodiments, a local particle swarm optimization algorithm is used in the high-throughput structural search, 35 to 50 structures are calculated for each iteration, and 20 to 30 iterations are calculated in total.

In one of the embodiments, the high-throughput structural search further includes calculation of structure relaxation, a cut-off energy of the structure relaxation is 400 ev to 500 ev, and a PBE functional in a generalized gradient approximation is used as a functional.

In one of the embodiments, before performing the structural search based on the first principle calculation, the method further includes determining a number range of each atom according to the species of the atoms in the components of the target glass.

In one of the embodiments, the step of comparing the formation energies and the phonon spectrums of the compounds respectively includes:

constructing a bump map to illustrate change of formation energies of the compounds with the components, and judging thermodynamically stable compounds in the compounds according to the bump map; and

calculating phonon spectrums of the thermodynamically stable compounds, and selecting a compound that does not contain an imaginary frequency in the phonon spectrum, which is namely the stable compound.

In one of the embodiments, the target glass includes one or more of a laser glass, a optical glass, a biological glass, a nuclear technology glass, a safety glass and a ware glass.

The present invention further provides a method for performance prediction of a ternary glass system, which includes the following steps:

combining any two of three components in the target glass to obtain three binary composition systems, and performing structural search on each of the binary composition systems respectively according to the method for performance prediction of the binary glass system to obtain corresponding stable compounds in each of the binary composition systems;

combining the three components of the target glass to obtain a ternary composition system, determining a proportion of four atoms in the ternary composition system, and performing structural search based on the first principle to search out compounds that can be formed by the four atoms in the ternary composition system;

comparing formation energies and phonon spectrums of the compounds that can be formed by the four atoms in the ternary composition system with formation energies and phonon spectrums of the stable compounds in the binary composition systems to determine stable compounds in the compounds that can be formed by the four atoms in the ternary composition system;

drawing a composition triangle by taking the components in the ternary composition system as vertexes, marking coordinates of all the stable compounds in the binary composition system and all the stable compounds in the ternary composition system in the composition triangle, taking the coordinates of all the stable compounds as vertexes, and dividing a triangular region according to a minimum area principle to obtain a ternary glass system composition diagram;

finding out a composition coordinate corresponding to the target glass in the ternary glass system composition diagram, microstructural units of glassy compounds corresponding to the compounds represented by three vertexes of the triangular region where the composition coordinate is located being structural genes of the target glass; and

calculating a property of the target glass according to a leverage model formula of the ternary glass system, the leverage model formula of the ternary glass system being P₀=P1×L1+P2×L2+P3×L3, wherein P₀ is the property of the target glass, P1, P2 and P3 are properties of the structural genes of the target glass, and L1, L2 and L3 are contents of the structural genes of the target glass.

In one of the embodiments, the step of comparing the formation energies and the phonon spectrums of the compounds that are formed by the four atoms in the ternary composition system with the formation energies and the phonon spectrums of the stable compounds in the binary composition system includes:

constructing a bump map illustrating the formation energies of the compounds that can be formed by the four atoms in the ternary composition system which change with the components by taking the stable compounds in the binary composition system as terminal vertexes of the components, and judging the thermodynamically stable compounds according to the bump map; and

calculating phonon spectrums of the thermodynamically stable compounds, and selecting a compound that does not contain an imaginary frequency in the phonon spectrum, which is namely the stable compound.

In one of the embodiments, when no stable compound exists in the compounds that are formed by the four atoms in the ternary composition system, all the stable compounds in the binary composition system are marked in the composition triangle only.

In one of the embodiments, when the stable compound exists in the compounds that are formed by the four atoms in the ternary composition system, all the stable compounds in the binary composition system and all the stable compounds in the ternary composition system are marked in the composition triangle.

The present invention further provides a method for predicting a density of a binary glass system, which includes the following steps:

performing structural search based on the first principle to search out compounds that can be formed between every two atoms or among every three atoms in components of a target glass, and obtaining formation energies and phonon spectrums of the compounds by calculating;

comparing the formation energies and the phonon spectrums of the compounds respectively to obtain stable compounds;

drawing a composition triangle by taking composition atoms of the target glass as vertexes, and marking coordinates of the stable compounds in the composition triangle to obtain a binary glass system composition diagram;

finding out a composition coordinate of the target glass in the binary glass system composition diagram, microstructural units of glassy compounds corresponding to two stable compounds adjacent to the composition coordinate are structural genes of the target glass; and

calculating a density of the target glass according to a leverage model formula of the binary glass system which is P₀=P1×L1+P2×L2, wherein P₀ is the density of the target glass, P1 and P2 are densities of the structural genes of the target glass, and L1 and L2 are contents of the structural genes of the target glass in the target glass.

The present invention further provides a method for predicting a refractive index of a binary glass system, which includes the following steps:

performing structural search based on the first principle to search out compounds that can be formed between every two atoms or among every three atoms in components of a target glass, and obtaining formation energies and phonon spectrums of the compounds by calculating;

comparing the formation energies and the phonon spectrums of the compounds respectively to obtain stable compounds;

drawing a composition triangle by taking composition atoms of the target glass as vertexes, and marking coordinates of the stable compounds in the composition triangle to obtain a binary glass system composition diagram;

finding out a composition coordinate of the target glass in the binary glass system composition diagram, microstructural units of glassy compounds corresponding to two stable compounds adjacent to the composition coordinate are structural genes of the target glass; and

calculating a refractive index of the target glass according to a leverage model formula of the binary glass system, the leverage model formula of the binary glass system being P₀=P1×L1+P2×L2, wherein P₀ is the refractive index of the target glass, P1 and P2 are refractive indexes of the structural genes of the target glass, and L1 and L2 are contents of the structural genes of the target glass in the target glass.

The present invention further provides a method for predicting a luminescent property of a binary glass system, which includes the following steps of:

performing structural search based on the first principle to search out compounds that can be formed between every two atoms or among every three atoms in components of a target glass, and obtaining formation energies and phonon spectrums of the compounds by calculating;

comparing the formation energies and the phonon spectrums of the compounds respectively to obtain stable compounds;

drawing a composition triangle by taking composition atoms of the target glass as vertexes, and marking coordinates of the stable compounds in the composition triangle to obtain a binary glass system composition diagram;

finding out a composition coordinate of the target glass in the binary glass system composition diagram, microstructural units of glassy compounds corresponding to two stable compounds adjacent to the composition coordinate are structural genes of the target glass; and

calculating a luminescent property of the target glass according to a leverage model formula of the binary glass system, the leverage model formula of the binary glass system being P₀=P1×L1+P2×L2, wherein P₀ is the luminescent property of the target glass, P1 and P2 are luminescent properties of the structural genes of the target glass, and L1 and L2 are contents of the structural genes of the target glass in the target glass.

The present invention further provides a method for predicting a density of a ternary glass system, which includes the following steps:

combining any two of three components of a target glass to obtain three binary composition systems, and performing structural search on each of the binary composition systems respectively according to the method for performance prediction of the binary glass system to obtain corresponding stable compounds in each of the binary composition systems;

combining the three components of the target glass to obtain a ternary composition system, determining a proportion of four atoms in the ternary composition system, and performing structural search based on the first principle to search out compounds that can be formed by the four atoms in the ternary composition system;

comparing formation energies and phonon spectrums of the compounds that can be formed by the four atoms in the ternary composition system with formation energies and phonon spectrums of the stable compounds in the binary composition systems to determine stable compounds in the compounds that can be formed by the four atoms in the ternary composition system;

drawing a composition triangle by taking the components in the ternary composition system as vertexes, marking coordinates of all the stable compounds in the binary composition system and all the stable compounds in the ternary composition system in the composition triangle, taking the coordinates of all the stable compounds as vertexes, and dividing a triangular region according to the minimum area principle to obtain a ternary glass system composition diagram;

finding out a composition coordinate corresponding to the target glass in the ternary glass system composition diagram, microstructural units of glassy compounds corresponding to the compounds represented by three vertexes of the triangular region where the composition coordinate is located being structural genes of the target glass; and

calculating a density of the target glass according to a leverage model formula of the ternary glass system, the leverage model formula of the ternary glass system being P₀=P1×L1+P2×L2+P3×L3, wherein P₀ is the density of the target glass, P1, P2 and P3 are densities of the structural genes of the target glass, and L1, L2 and L3 are contents of the structural genes of the target glass in the target glass.

The present invention further provides a method for predicting a refractive index of a ternary glass system, which includes the following steps:

combining any two of three components of a target glass to obtain three binary composition systems, and performing structural search on each of the binary composition systems respectively according to the method for performance prediction of the binary glass system to obtain corresponding stable compounds in each of the binary composition systems;

combining the three components of the target glass to obtain a ternary composition system, determining a proportion of four atoms in the ternary composition system, and performing structural search based on the first principle to search out compounds that can be formed by the four atoms in the ternary composition system;

comparing formation energies and phonon spectrums of the compounds that can be formed by the four atoms in the ternary composition system with formation energies and phonon spectrums of the stable compounds in the binary composition systems to determine stable compounds in the compounds that can be formed by the four atoms in the ternary composition system;

drawing a composition triangle by taking the components in the ternary composition system as vertexes, marking coordinates of all the stable compounds in the binary composition system and all the stable compounds in the ternary composition system in the composition triangle, taking the coordinates of all the stable compounds as vertexes, and dividing a triangular region according to the minimum area principle to obtain a ternary glass system composition diagram;

finding out a composition coordinate corresponding to the target glass in the ternary glass system composition diagram, microstructural units of glassy compounds corresponding to the compounds represented by three vertexes of the triangular region where the composition coordinate is located being structural genes of the target glass; and

calculating a refractive index of the target glass according to a leverage model formula of the ternary glass system, the leverage model formula of the ternary glass system being P₀=P1×L1+P2×L2+P3×L3, wherein P₀ is the refractive index of the target glass, P1, P2 and P3 are refractive indexes of the structural genes of the target glass, and L1, L2 and L3 are contents of the structural genes of the target glass in the target glass.

The present invention further provides a method for predicting a luminescent property of a ternary glass system, which includes the following steps:

combining any two of three components of a target glass to obtain three binary composition systems, and performing structural search on each of the binary composition system respectively according to the method for performance prediction of the binary glass system to obtain corresponding stable compounds in each of the binary composition systems;

combining the three components of the target glass to obtain a ternary composition system, determining a proportion of four atoms in the ternary composition system, and performing structural search based on the first principle to search out compounds that can be formed by the four atoms in the ternary composition system;

comparing formation energies and phonon spectrums of the compounds that can be formed by the four atoms in the ternary composition system with formation energies and phonon spectrums of the stable compounds in the binary composition systems to determine stable compounds in the compounds that can be formed by the four atoms in the ternary composition system;

drawing a composition triangle by taking the components in the ternary composition system as vertexes, marking coordinates of all the stable compounds in the binary composition system and all the stable compounds in the ternary composition system in the composition triangle, taking the coordinates of all the stable compounds as vertexes, and dividing a triangular region according to the minimum area principle to obtain a ternary glass system composition diagram;

finding out a composition coordinate corresponding to the target glass in the ternary glass system composition diagram, microstructural units of glassy compounds corresponding to the compounds represented by three vertexes of the triangular region where the composition coordinate is located being structural genes of the target glass; and

calculating a luminescent property of the target glass according to a leverage model formula of the ternary glass system, the leverage model formula of the ternary glass system being P₀=P1×L1+P2×L2+P3×L3, wherein P₀ is the luminescent property of the target glass, P1, P2 and P3 are luminescent properties of the structural genes of the target glass, and L1, L2 and L3 are contents of the structural genes of the target glass in the target glass.

Compared with the prior art, the present invention has the following advantages.

(1) The present invention applies the material genome research concept to glass research, and innovatively proposes a method for researching and developing a glass material by multi-scale method, which, from an atomic level and based on the density functional theory, firstly proposes quantitative research on the properties of the glass materials realized by multi-scale method of combined atom-compound-glass structure and property from a micro level to a macro level. The structural search based on the first principle and the drawing of the composition triangle are combined to obtain the glass system composition diagram, the structural genes of the target glass are found using the glass system composition diagram, and the property of the target glass is quantitatively researched and predicted using the leverage model formula according to properties of the structural genes of the glass system, which is of great significance to the research and development of a functional glass, a special optical fiber and a fiber laser thereof.

(2) The multi-scale method proposed in the present invention is a method from a micro level to a macro level, through which rapid demand-based design of the binary, ternary and multiplex glass system materials can be realized. Compared with the traditional trial-and-error method for researching glass, the method has the advantages of a short test period, low costs, a high efficiency, etc.

(3) The present invention seeks the structural genes of the glass system from an atomic perspective according to the first principle, uses the microstructural units of the glassy compounds corresponding to two stable compounds adjacent to the composition coordinate of the target glass in the glass system composition diagram or the compounds represented by three vertexes of the triangular region where the composition coordinate is located as the structural genes of the target glass system, and predicts the property of the target glass by the found glass structural genes. Compared with a previous method of adding all oxides, the glassy compounds are closer to a real structure of a glass matrix, contain coordination polyhedron structural groups (structural genes) identical to the target glass, can reflect the structure and the property of the target glass, and can more accurately predict the density, the refractive index and the luminescent property of the target glass.

(4) According to the method for performance prediction of the glass provided by the present invention, the glass system composition diagram is drawn from the atomic perspective according to the first principle, and the structural genes of the target glass are sought using the glass system composition diagram, so that the property of the glass is predicted using the structural genes of the glass system, thus solving the problems of lack of phase diagram data and how the property of the glass is predicted for the glass system with a test error are solved, and realizing wider, accurate and efficient research and development of the glass system materials in a wider range.

### BRIEF DESCRIPTION OF THE DRAWINGS

FIG. 1 is a bump map illustrating relative formation energies of stable compounds of a Li₂O—GeO₂ binary glass system that change with components according to Embodiment 1 of the present invention;

FIG. 2 is a composition diagram of the Li₂O—GeO₂ binary glass system according to Embodiment 1 of the present invention;

FIG. 3 is a comparison diagram illustrating predicted values and test values of densities and refractive indexes of Li₂O—GeO₂ and Na₂O—GeO₂ binary glass systems according to Embodiment 1 and Embodiment 2 of the present invention;

FIG. 4 is a composition diagram of a Na₂O—GeO₂ binary glass system according to Embodiment 2 of the present invention; and

FIG. 5 is a composition diagram of a GeO₂—BaO—La₂O₃ ternary glass system according to Embodiment 3 of the present invention.

### DETAILED DESCRIPTION OF THE PREFERRED EMBODIMENT

To make the objectives, technical solutions, and advantages of the present invention clearer, the present invention is further described in detail hereinafter with reference to the embodiments and the accompanying drawings. It should be understood that the specific embodiments described herein are merely used to explain the present invention, but are not intended to limit the present invention.

An embodiment of the present invention provides a method for performance prediction of a multiplex glass system, which includes the following steps:

S01: determining species of atoms for structural search according to components of the multiplex glass system;

S02: performing structural search based on the first principle to search out compounds that can be formed by interaction between the atoms;

S03: comparing a formation energy and a phonon spectrum of each of the compound to obtain stable compounds;

S04: constructing a glass structural composition diagram according to the stable compounds, microstructural units of glassy compounds adjacent to a composition point of a target glass being structural genes of the glass; and

S05: calculating a property of the target glass according to a leverage model formula of the multiplex glass system, the leverage model formula of the multiplex glass system being P₀=Σᵢ₌₁ⁿPi×Li, wherein the multiplex glass system has n components, P₀ is the property of the target glass, Pi is a property of the structural gene of the target glass, and Li is a content of the structural gene of the target glass in the target glass.

The method for performance prediction of the multiplex glass system provided by the present invention is rapid and efficient. According to a short range order characteristic of the glass, the present invention innovatively proposes a concept of researching and developing a functional glass by multi-scale method of atom-compound-glass firstly, and predicts the property of the glass system from a micro level to a macro level, which, from an atomic level, seeks the structural genes of the glass system based on the first principle, and the property of the target glass is quantitatively researched by the structural genes of the glass system and the leverage model formula, which is of great significance to rapid, low-cost and efficient research and development of the functional glass, the special optical fiber and the fiber laser thereof.

In the present invention, the multiplex glass system is composed of a plurality of oxides, and the components are the oxides forming the multiplex glass system. The binary glass system includes two components, and the ternary glass system includes three components. For example, components of a Li₂O—GeO₂ binary glass system are Li₂O and GeO₂, and components of a BaO—La₂O₃.GeO₂ ternary glass system are BaO, La₂O₃ and GeO₂.

In the present invention, the compound includes a plurality of different compounds, which include compounds with different atomic compositions, and also include compounds with the same atomic composition but different structures.

An embodiment of the present invention provides a method for performance prediction of a binary glass system, which includes the following steps:

S10: performing structural search based on the first principle to search out compounds that can be formed between every two atoms or among every three atoms in components of a target glass, and obtaining formation energies and phonon spectrums of the compounds by calculating;

S20: comparing the formation energies and the phonon spectrums of the compounds respectively to obtain stable compounds;

S30: drawing a composition triangle by taking composition atoms of the target glass as vertexes, and marking coordinates of the stable compounds in the composition triangle to obtain a binary glass system composition diagram;

S40: finding out a composition coordinate of the target glass in the binary glass system composition diagram, microstructural units of glassy compounds corresponding to two stable compounds adjacent to the composition coordinate are structural genes of the target glass; and

S50: calculating a property of the target glass according to a leverage model formula of the binary glass system, the leverage model formula of the binary glass system is P₀=P1×L1+P2×L2, wherein P₀ is the property of the target glass, P1 and P2 are properties of the structural genes of the target glass, and L1 and L2 are contents of the structural genes of the target glass in the target glass.

The property may be at least one of a mechanical property, a magnetic property, an electrical property, a luminescent property and a thermal property. In an embodiment, the property is at least one of a density, a refractive index, a fluorescence full width at half maximum, an effective line width, an absorption cross-section and a peak emission cross-section.

The structural search based on the first principle is that the compounds that can be formed can be obtained according to properties of the atoms, which is an ab initio calculation algorithm.

In an embodiment, performing the structural search based on the first principle is to perform high-throughput structural search using the first principle structural search software, such as CALYPSO and VASP. The high-throughput structural search is performed in a concurrent mode, which is beneficial for accelerating a searching efficiency.

In an embodiment, a local particle swarm optimization algorithm is used in the high-throughput structural search, 35 to 50 structures are calculated for each iteration, and 20 to 30 iterations are calculated in total.

In an embodiment, the high-throughput structural search further includes structure relaxation calculation, a cut-off energy of the structure relaxation is 400 ev to 500 ev, and a PBE functional in a generalized gradient approximation (GGA) is used as a functional.

In an embodiment, before performing the structural search based on the first principle, the method further includes a step S00 of determining a number range of each atom for structural search according to species of the atoms in the components of the target glass.

In an embodiment, the step S20 of comparing the formation energies and the phonon spectrums of the compounds respectively includes:

S22: constructing a bump map illustrating the formation energies of the compounds obtained by calculating which change with the components, and judging thermodynamically stable compounds in the compounds according to the bump map; and

S24: calculating phonon spectrums of the thermodynamically stable compounds, and selecting a compound that does not contain an imaginary frequency (i.e. dynamically stable compound) in the phonon spectrum, which is namely the stable compound.

In the step S30, the composition triangle is a triangle drawn according to a component representation method of a multiplex phase diagram, which may also be referred to as a concentration triangle. A parallel line of each side is respectively made passing through any point in the composition triangle, and a line segment cut by the parallel line of each side of the composition triangle respectively represents a concentration or a proportion of each component at the point. The coordinates are points corresponding to a compound of a specific composition in the composition triangle. In the step S40, the composition coordinate is a point corresponding to the component of the target glass in the composition triangle.

In an embodiment, the target glass includes one or more of a laser glass, an optical glass, a biological glass, a nuclear technology glass, a safety glass and a ware glass.

An embodiment of the present invention further provides a method for performance prediction of a ternary glass system, which includes the following steps:

S100: combining any two of three components of a target glass to obtain three binary composition systems, and performing structural search on each of the binary composition systems respectively according to the method for performance prediction of a binary glass system to obtain corresponding stable compounds in each of the binary composition systems;

S200: combining the three components of the target glass to obtain a ternary composition system, determining a proportion of four atoms in the ternary composition system, and performing structural search based on the first principle to search out compounds that can be formed by the four atoms in the ternary composition system;

S300: comparing formation energies and phonon spectrums of the compounds that can be formed by the four atoms in the ternary composition system with formation energies and phonon spectrums of the stable compounds in the binary composition systems to determine stable compounds in the compounds that can be formed by the four atoms in the ternary composition system;

S400: drawing a composition triangle by taking the components in the ternary composition system as vertexes, marking coordinates of all the stable compounds in the binary composition system and all the stable compounds in the ternary composition system in the composition triangle, taking the coordinates of all the stable compounds as vertexes, and dividing a triangular region according to the minimum area principle to obtain a ternary glass system composition diagram;

S500: finding out a composition coordinate corresponding to the target glass in the ternary glass system composition diagram, microstructural units of glassy compounds corresponding to the compounds represented by three vertexes of the triangular region where the composition coordinate is located are structural genes of the target glass; and

S600: calculating a property of the target glass according to a leverage model formula of the ternary glass system, the leverage model formula of the ternary glass system being P₀=P1×L1+P2×L2+P3×L3, wherein P₀ is the property of the target glass, P1, P2 and P3 are properties of the structural genes of the target glass, and L1, L2 and L3 are contents of the structural genes of the target glass in the target glass.

In an embodiment, the step S300 of comparing the formation energies and the phonon spectrums of the compounds that can be formed by the four atoms in the ternary composition system with the formation energies and the phonon spectrums of the stable compounds in the binary composition system includes:

S320: constructing a bump map illustrating the formation energies of the compounds that can be formed by the four atoms in the ternary composition system which change with the components by taking the stable compounds in the binary composition system as terminal vertexes of the components, and judging the thermodynamically stable compounds according to the bump map; and

S340: calculating phonon spectrums of the thermodynamically stable compounds, and selecting a compound that does not contain an imaginary frequency in the phonon spectrum, which is namely the stable compound.

In an embodiment, when no stable compound exists in the compounds that can be formed by the four atoms in the ternary composition system, all the stable compounds in the binary composition system are marked in the composition triangle only in the step S400.

In an embodiment, when the stable compound exists in the compounds that can be formed by the four atoms in the ternary composition system, all the stable compounds in the binary composition system and all the stable compounds in the ternary composition system are marked in the composition triangle in the step S400.

According to the method for performance prediction of the glass system provided by the embodiment of the present invention, a biological gene concept and a material genome engineering research mode are used for reference to seek the structural genes of the glass system, a sequential iteration method in a traditional trial-and-error method is replaced by a high-throughput concurrent iteration method, and the “experience-guided experiment” is changed to the “combination of theoretical prediction and test verification” in a research and development mode of materials, so as to realize a target of “shortening a research and development cycle by half and reducing research and development costs by half” and accelerate a process of “discovery-development-production-application” of new materials.

The binary glass system composition diagram and the ternary glass system composition diagram reflect the glass composition, and the glass composition points can be corresponded one by one in the diagrams. In the binary glass system composition diagram and the ternary glass system composition diagram, the microstructural units of the glassy compounds corresponding to the two stable compounds adjacent to the composition coordinate of the target glass or the compounds represented by the three vertexes of the triangular region where the composition coordinate is located are the structural genes of the glass system.

The structural genes of the glass system contain a polyhedral coordination condition identical to the target glass, reflect the structure of the glass and determine the property of the glass. The composition points of the glass system can be corresponded one by one in the glass system composition diagram.

Germanate glass has attracted much attention in the field of mid-infrared fiber lasers because of having advantages such as good mid-infrared transmittance, low phonon energy, and high solubility of rare earth ions. Li₂O—GeO₂ and Na₂O—GeO₂ glass systems are researched by methods for predicting a density and a refractive index of the binary glass system provided by the present invention in the following embodiments.

### Embodiment 1 Li₂O—GeO₂ Binary Glass System

Target glass was: x mol % Li₂O-y mol % GeO₂

A number range of each of Ge, Li and O atoms was set, wherein a number of Ge atoms ranged from 0 to 8, a number of Li atoms ranged from 0 to 8, and a number of 0 atoms ranged from 1 to 10.

According to a number ratio of every two or three atoms, structural search was performed in a first principle structural search software CALYPSO, a local particle swarm optimization algorithm was used for structure evolution, and 35 structures were generated in each iteration. Structure relaxation was performed on a structure screened out by a first principle calculation software VASP, cut-off energy was 500 ev, and a PBE functional in a generalized gradient approximation (GGA) was used as a functional. Compounds that can be formed including GeO₂, Li₂O.7GeO₂, Li₂O.4GeO₂, 3Li₂O.8GeO₂, Li₂O.2GeO₂ and Li₂O were obtained.

Formation energies of the compounds were also obtained.

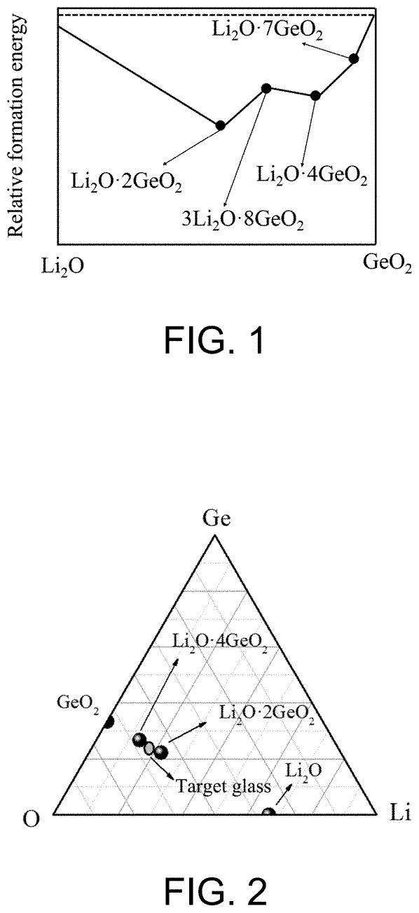

A bump map illustrating the formation energies changed with the components was constructed based on the formation energies of the compounds, which was shown in FIG. 1. Thermodynamically stable compounds in the compounds were judged to be GeO₂, Li₂O.7GeO₂, Li₂O.4GeO₂, Li₂O.2GeO₂ and Li₂O according to the bump map.

Phonon spectrums of the thermodynamically stable compounds were calculated, and compounds that did not contain an imaginary frequency in the phonon spectrums were selected, which were namely the stable compounds, including GeO₂, Li₂O.4GeO₂, Li₂O.2GeO₂ and Li₂O.

A composition triangle was drawn by taking atoms Ge, Li and O as vertexes, and coordinates of GeO₂, Li₂O.4GeO₂, Li₂O.2GeO₂ and Li₂O were marked in the composition triangle to obtain a Li₂O—GeO₂ binary glass system composition diagram as shown in FIG. 2.

When x was 25.7 and y was 74.3, i.e., the target glass was 25.7 mol % Li₂O-74.3 mol % GeO₂, a composition coordinate of the target glass was found in FIG. 2. The coordinate was located between Li₂O.2GeO₂ and Li₂O.4GeO₂ in FIG. 2, and glassy Li₂O.2GeO₂ and Li₂O.4GeO₂ were structural genes of the glass formed by 25.7 mol % Li₂O-74.3 mol % GeO₂.

A density and a refractive index of the above-described target glass were calculated according to a leverage model formula of the binary glass system P₀=P1×L1+P2×L2, wherein P1 was a density or a refractive index of Li₂O.2GeO₂, P2 was a density or a refractive index of Li₂O.4GeO₂, and a density and a refractive index of each glassy compound in Li₂O—GeO₂ and Na₂O—GeO₂ binary glass systems were obtained by experiments, which were shown in Table 1. L1 was a content of Li₂O.2GeO₂ in the target glass, L2 was a content of Li₂O.4GeO₂ in the target glass. L1 was 42.75 and L2 was 57.25 by calculating. The densities of Li₂O.2GeO₂ and Li₂O.4GeO₂ in the Table, i.e. P1 and P2, were substituted into the formula to obtain that P₀=3.8523, and an experimental value of the density of the target glass 25.7 mol % Li₂O-74.3 mol % GeO₂ was 3.8612. Similarly, the refractive indexes of Li₂O.4GeO₂ and Li₂O.2GeO₂ in Table 1 were substituted into the formula as P1 and P2 to obtain that a predicted value of the refractive index of the target glass 25.7 mol % Li₂O-74.3 mol % GeO₂ was 1.694 by calculating.

TABLE 1

| Glassy compound | Density (g/cm³) | Refractive index |
|---|---|---|
| GeO₂ | 3.667 | 1.608 |
| Li₂O•2GeO₂ | 3.51 | 1.657 |
| Li₂O•4GeO₂ | 4.108 | 1.721 |
| 2Na₂O•9GeO₂ | 4.10 | 1.683 |
| Na₂O•2GeO₂ | 3.58 | 1.630 |
| Na₂O•GeO₂ | 3.31 | — |

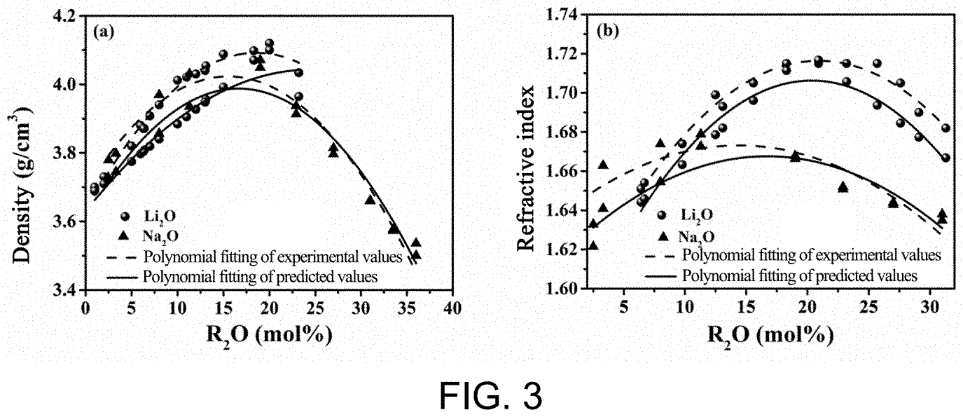

When a series of different values of x and y were taken, predicted values of densities and predicted values of refractive indexes of Li₂O—GeO₂ binary glass systems with various compositions were calculated and compared with densities and refractive indexes of Li₂O—GeO₂ binary glass systems with corresponding compositions obtained by experiments. Results were shown in FIG. 3. It can be seen from FIG. 3 that the predicted values of the densities and the predicted values of the refractive indexes of the Li₂O—GeO₂ binary glass systems calculated according to the above-described methods have an relative error within 5% in comparison to the experimental values, which demonstrates that the methods for predicting the density and the refractive index of the binary glass system are effective. The density of the glass was measured by a drainage method, and the refractive index was measured by a Metricon 2010 prism coupler.

### Embodiment 2 Na₂O—GeO₂ Binary Glass System

Target glass was: x mol % Na₂O-y mol % GeO₂

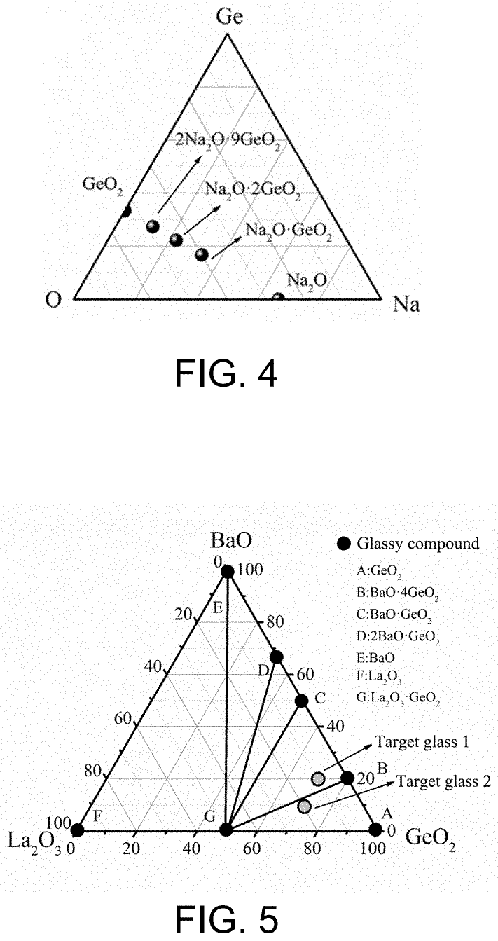

Methods for predicting a density and a refractive index of the Na₂O—GeO₂ binary glass system were basically the same as those in Embodiment 1, except that the glass system was different, and a glass system composition diagram thereof was shown in FIG. 4.

When a series of different values of x and y were taken, predicted values of densities and predicted values of refractive indexes of Na₂O—GeO₂ binary glass systems with various compositions were calculated and compared with densities and refractive indexes of Na₂O—GeO₂ binary glass systems with corresponding compositions obtained by experiments. Results were shown in FIG. 3. It can be seen from FIG. 3 that the predicted values of the densities and the predicted values of the refractive indexes of the Na₂O—GeO₂ binary glass systems calculated according to the above-described methods have an relative error within 5% in comparison to the experimental values, which demonstrates that the methods for predicting the density and the refractive index of the binary glass system are effective. The density of the glass was measured by a drainage method, and the refractive index was measured by a Metricon 2010 prism coupler.

### Embodiment 3 GeO₂—BaO—La₂O₃ Ternary Glass System

Target glass was: x mol % GeO₂-y mol % BaO-z mol % La₂O₃ (x≥56 mol %, y≤50 mol % and z≤20 mol %)

The GeO₂—BaO—La₂O₃ glass system is an important germanate glass matrix material. The germanate glass has attracted much attention in the field of mid-infrared fiber lasers because of having advantages such as good mid-infrared transmittance, low phonon energy, and high solubility of rare earth ions, and is an important laser glass material. The GeO₂—BaO—La₂O₃ glass system was researched by the method for performance prediction of the ternary glass system in the following embodiment.

Any two of components GeO₂, BaO and La₂O₃ were combined to obtain a GeO₂—BaO binary composition system, a GeO₂—La₂O₃ binary composition system and a BaO—La₂O₃ binary composition system. According to the steps S00 to S40, stable compounds in the GeO₂—BaO binary composition system, including GeO₂, BaO.4GeO₂, BaO.GeO₂, 2BaO.GeO₂ and BaO, and stable compounds in the GeO₂—La₂O₃ binary composition system, including La₂O₃ and La₂O₃.GeO₂, were obtained respectively, and no stable compound existed in the BaO—La₂O₃ binary composition system.

The components GeO₂, BaO and La₂O₃ were combined to obtain a GeO₂—BaO—La₂O₃ ternary composition system. For the four atoms in the ternary composition system, a range of the structural search was determined to be Ge: 1-5, Ba: 1-5, La: 1-5, O: 1-10. First principle structural search software and calculation software were used to perform high-throughput structural search to search out compounds that can be formed by the atoms Ge, Ba, La and O, and calculate formation energies and phonon spectrums of the compounds.

The formation energies and the phonon spectrums of the compounds that can be formed by atoms Ge, Ba, La and O were compared with the formation energies and the phonon spectrums of GeO₂, BaO.4GeO₂, BaO.GeO₂, 2BaO.GeO₂, BaO, La₂O₃ and La₂O₃.GeO₂. According to comparison results, no stable compound existed in the compounds that can be formed by the atoms Ge, Ba, La and O.

A composition triangle was drawn by taking GeO₂, BaO and La₂O₃ as vertexes, coordinates of all the stable compounds (A: GeO₂, B: BaO.4GeO₂, C: BaO.GeO₂, D: 2BaO.GeO₂, E: BaO, F: La₂O₃, and G: La₂O₃.GeO₂) were marked in the composition triangle. Using A, B, C, D, E, F, G as vertexes, a triangular region was divided according to the minimum area principle to obtain a ternary glass system composition diagram, as shown in FIG. 5.

When x was 70, y was 20 and z was 10, i.e., a target glass 1 was 70 mol % GeO₂-20 mol % BaO-10 mol % La₂O₃, a composition coordinate of the target glass 1 was found in FIG. 5. The coordinate was located in ΔBCG, and structural genes of the target glass were glassy BaO.4GeO₂, BaO.GeO₂ and La₂O₃.GeO₂.

A density of the target glass above was calculated according to a leverage model formula of the ternary glass system P₀=P1×L1+P2×L2+P3×L3, wherein P1 was a density of BaO.4GeO₂, P2 was a density of BaO.GeO₂, P3 was a density of La₂O₃.GeO₂. A density of each glassy compound in the GeO₂—BaO—La₂O₃ ternary glass system obtained by experiments was as shown in Table 2. It can be seen from Table 2 that P1 was 5.15 g/cm³, P2 was 5.06 g/cm³, and P3 was 5.88 g/cm³. L1 was a content of BaO.4GeO₂ in the target glass, which was 66.67% by calculating, L2 was a content of BaO.GeO₂ in the target glass, which was 13.33% by calculating, and L3 was a content of La₂O₃.GeO₂ in the target glass, which was 20% by calculating. The values were substituted into the formula P₀=5.15 g/cm³×66.67%+5.06 g/cm³×13.33%+5.88 g/cm³×20%=5.231 g/cm³.

TABLE 2

| Glassy compound | Density (g/cm³) |
|---|---|
| BaO | 5.72 |
| GeO₂ | 3.667 |
| BaO•4GeO₂ | 5.15 |
| BaO•GeO₂ | 5.06 |
| 2BaO•GeO₂ | 5.8 |
| La₂O₃•GeO₂ | 5.88 |
| La₂O₃ | 6.57 |

When a series of different values of x, y and z were taken, predicted values of densities of GeO₂—BaO—La₂O₃ ternary glass systems with various compositions were calculated and compared with densities of GeO₂—BaO—La₂O₃ ternary glass systems with corresponding compositions obtained by experiments to calculate relative errors. Results were shown in Table 3. It can be seen from Table 3 that the predicted values of the glass systems calculated according to the above-described method have a relative error within 5% in comparison with experimental values, which demonstrates that the method for predicting the density of the ternary glass system is effective.

TABLE 3

| Glass composition | | | Glassy compound composition | | | Density (g/cm³) | | |
|---|---|---|---|---|---|---|---|---|
| (mol %) | | | (mol %) | | | Experimental | Predicted | Relative |
| GeO₂ | BaO | La₂O₃ | BaO•4GeO₂ | BaO•GeO₂ | La₂O₃•GeO₂ | value | value | error (%) |
| 70 | 20 | 10 | 66.67 | 13.33 | 20 | 5.092 | 5.231 | 2.72 |
| 55 | 40 | 5 | 16.67 | 73.33 | 10 | 5.038 | 5.144 | 2.10 |
| 65 | 30 | 5 | 50 | 40 | 10 | 5.012 | 5.147 | 2.69 |
| 75 | 20 | 5 | 83.33 | 6.67 | 10 | 4.963 | 5.150 | 3.77 |
| 65 | 20 | 15 | 50 | 20 | 30 | 5.222 | 5.311 | 1.70 |
| 65 | 25 | 10 | 50 | 30 | 20 | 5.092 | 5.229 | 2.69 |
| 55 | 35 | 10 | 16.67 | 63.33 | 20 | 5.157 | 5.226 | 1.33 |
| 72.5 | 20 | 7.5 | 75 | 10 | 15 | 5.021 | 5.191 | 3.38 |
| 67.5 | 25 | 7.5 | 58.33 | 26.67 | 15 | 5.041 | 5.189 | 2.93 |
| 67.5 | 20 | 12.5 | 58.33 | 16.67 | 25 | 5.156 | 5.271 | 2.23 |
| 50 | 45 | 5 | 0 | 90 | 10 | 5.1 | 5.142 | 0.82 |
| | | | GeO₂ | BaO•4GeO₂ | La₂O₃•GeO₂ | | | |
| 85 | 10 | 5 | 40 | 50 | 10 | 4.751 | 4.630 | −2.55 |
| 80 | 5 | 15 | 45 | 25 | 30 | 5.141 | 4.702 | −8.55 |
| 75 | 15 | 10 | 5 | 75 | 20 | 5.042 | 5.222 | 3.57 |
| 82.5 | 10 | 7.5 | 35 | 50 | 15 | 4.882 | 4.740 | −2.90 |
| 77.5 | 10 | 12.5 | 25 | 50 | 25 | 5.12 | 4.962 | −3.09 |

Based on the above-described method for predicting the density, refractive indexes of the GeO₂—BaO—La₂O₃ ternary glass systems with various compositions can also be effectively predicted by replacing the density with the refractive index. The density of the glass was measured by a drainage method, and the refractive index was measured by a Metricon 2010 prism coupler.

### Embodiment 4

Target glass was: x mol % GeO₂-y mol % BaO-z mol % La₂O₃-(1-x-y-z)Tm₂O₃ (x≥56 mol %, y≤50 mol % and z≤20 mol %)

Tm³⁺ doped germanate glass, xGeO₂-yBaO-zLa₂O₃-(1-x-y-z)Tm₂O₃ (x≥56 mol %, y≤50 mol % and z≤20 mol %), is an important laser glass material. A luminescent property of the Tm³⁺ doped xGeO₂-yBaO-zLa₂O₃ glass was researched according to the method for performance prediction of the ternary glass system. The luminescent property is a luminescent property of ³F₄→³H₆ energy level transition of Tm³⁺ ions, including a fluorescence full width at half maximum, an effective line width and a peak emission cross-section of ³F₄→³H₆ transition of rare earth ion Tm³⁺ ions in the glass, as well as absorption cross-sections of the Tm³⁺ ion at 790 nm and 1610 nm.

Tm₂O₃ was used as a doping component, and the xGeO₂-yBaO-zLa₂O₃-(1-x-y-z)Tm₂O₃ glass system was equivalent to the GeO₂—BaO—La₂O₃ ternary glass system. An x mol % GeO₂-y mol % BaO-z mol % La₂O₃-(1-x-y-z)Tm₂O₃ glass system composition diagram was obtained based on the same method in the Embodiment 3, as shown in FIG. 5. Similarly, structural genes of the target glass were found based on the same method. Structural genes of a target glass 2 which was 69.2 mol % GeO₂-10 mol % BaO-20 mol % La₂O₃-0.8 mol % Tm₂O₃ were glassy GeO₂, BaO.4GeO₂ and La₂O₃.GeO₂.

The luminescent property of the above-described target glass was calculated according to a leverage model formula of the ternary glass system P₀=P1×L1+P2×L2+P3×L3, wherein P1 was a luminescent property of GeO₂, P2 was a luminescent property of BaO.4GeO₂, and P3 was a luminescent property of La₂O₃.GeO₂; and L1 was a content of GeO₂ in the target glass, which was 9.2% by calculating, L2 was a content of BaO.4GeO₂ in the target glass, which was 50% by calculating, and L3 was a content of La₂O₃.GeO₂ in the target glass, which was 40% by calculating. a luminescence property, a fluorescence full width at half maximum, an effective line width, an absorption cross-section at 790 nm, an absorption cross-section at 1610 nm, and a peak emission cross-section of each glassy compound in the Tm₂O₃ doped GeO₂—BaO—La₂O₃ ternary glass system obtained by experiments were as shown in Table 4. Luminescence property data of each glassy compound in Table 4 were substituted into the formula to obtain a fluorescence full width at half maximum, an effective line width, an absorption cross-section at 790 nm, an absorption cross-section at 1610 nm, and a peak emission cross-section of the 69.2 mol % GeO₂-10 mol % BaO-20 mol % La₂O₃-0.8 mol % Tm₂O₃ glass system by calculating.

TABLE 4

| | Fluorescence | | Absorption | Absorption | Peak emission |
|---|---|---|---|---|---|
| | full width at half | Effective | cross-section at | cross-section at | cross-section |
| Glassy | maximum | line width | 790 nm (10⁻²¹ | 1610 nm (10⁻²¹ | (10⁻²¹ |
| compound | (FWHM) | (nm) | cm²) | cm²) | cm²) |
| GeO₂ | 238.16 | 246.16 | 9.33 | 3.09 | 6.77 |
| BaO•4GeO₂ | 230.35 | 260.42 | 6.72 | 2.48 | 4.60 |
| BaO•GeO₂ | 213.06 | 250.26 | 3.78 | 1.32 | 2.94 |
| La₂O₃•GeO₂ | 345.81 | 307.45 | 8.64 | 4.53 | 8.38 |

Similarly, luminescence properties of x mol % GeO₂-y mol % BaO-z mol % La₂O₃-(1-x-y-z)Tm₂O₃ glass systems with various compositions were calculated based on the same method when a series of different values of x, y and z were taken, and were compared with luminescence properties of glasses with corresponding compositions obtained by experiments to calculate relative errors. Results were shown in Tables 5 and Table 6. It can be seen from Table 5 and Table 6 that the predicted values of the glass systems calculated by the method above have a relative error within 11% compared with experimental values, which demonstrates that the method for predicting the luminescent property of the ternary glass system is effective.

TABLE 5

| | | | | | | Fluorescence full | | | | | |
|---|---|---|---|---|---|---|---|---|---|---|---|
| | | | | | | width at half | | | Effective line width | | |
| Glass oxide | | | Glassy compound composition | | | maximum (FWHM) | | | (nm) | | |
| composition | | | of congruent fusion | | | | | Relative | | | Relative |
| (mol %) | | | (mol %) | | | Experimental | Predicted | error | Experimental | Predicted | error |
| GeO₂ | BaO | La₂O₃ | GeO₂ | BaO•4GeO₂ | La₂O₃•GeO₂ | value | value | (%) | value | value | (%) |
| 69.2 | 10 | 20 | 9.2 | 50 | 40 | 252.64 | 275.41 | 9.01 | 263.68 | 275.83 | 4.61 |
| 79.2 | 5 | 15 | 44.2 | 25 | 30 | 250.25 | 266.59 | 6.53 | 259.89 | 266.14 | 2.41 |
| 84.2 | 10 | 5 | 39.2 | 50 | 10 | 246.41 | 243.11 | −1.34 | 261.38 | 257.45 | −1.50 |
| 79.2 | 15 | 5 | 14.2 | 75 | 10 | 248.66 | 241.16 | −3.02 | 264.08 | 261.01 | −1.16 |
| | | | BaO•4GeO₂ | BaO•GeO₂ | La₂O₃•GeO₂ | | | | | | |
| 69.2 | 20 | 10 | 65.33 | 13.87 | 20 | 245.06 | 249.20 | 1.69 | 260.66 | 266.33 | 2.18 |
| 59.2 | 30 | 10 | 32 | 47.2 | 20 | 244.97 | 243.43 | −0.63 | 264.46 | 262.94 | −0.57 |
| 59.2 | 25 | 15 | 32 | 37.2 | 30 | 251.34 | 256.71 | 2.14 | 266.97 | 268.66 | 0.64 |
| 69.2 | 15 | 15 | 65.33 | 3.87 | 30 | 251.92 | 262.47 | 4.19 | 264.41 | 272.05 | 2.89 |

TABLE 6

| | | | | | | Absorption cross- | | | Absorption cross- | | | Peak emission | | |
|---|---|---|---|---|---|---|---|---|---|---|---|---|---|---|
| | | | | | | section at 790 nm | | | section at 1610 nm | | | cross-section | | |
| | | | | | | (10⁻²¹ cm²) | | | (10⁻²¹ cm²) | | | (10⁻²¹ cm²) | | |
| Glass oxide | | | Glassy compound | | | | | Rela- | | | Rela- | | | Rela- |
| composition | | | composition | | | Exper- | Pre- | tive | Exper- | Pre- | tive | Exper- | Pre- | tive |
| (mol %) | | | (mol %) | | | imental | dicted | error | imental | dicted | error | imental | dicted | error |
| GeO₂ | BaO | La₂O₃ | GeO₂ | BaO•4GeO₂ | La₂O₃•GeO₂ | value | value | (%) | value | value | (%) | value | value | (%) |
| 79.2 | 10 | 10 | 29.2 | 50 | 20 | 7.29 | 7.81 | 7.15 | 2.86 | 3.05 | 6.60 | 5.38 | 5.95 | 10.63 |
| 69.2 | 10 | 20 | 9.2 | 50 | 40 | 7.13 | 7.67 | 7.62 | 3.11 | 3.34 | 7.31 | — | 6.27 | |
| 79.2 | 5 | 15 | 44.2 | 25 | 30 | 8.12 | 8.40 | 3.41 | 3.23 | 3.35 | 3.56 | 6.21 | 6.65 | 7.15 |
| 84.2 | 10 | 5 | 39.2 | 50 | 10 | 8.16 | 7.88 | 3.45 | 3.17 | 2.90 | 8.24 | 5.83 | 5.79 | 0.67 |
| 79.2 | 15 | 5 | 14.2 | 75 | 10 | 7.42 | 7.23 | 2.63 | 2.79 | 2.75 | 1.28 | 5.18 | 5.25 | 1.35 |
| | | | BaO•4GeO₂ | BaO•GeO₂ | La₂O₃•GeO₂ | | | | | | | | | |
| 59.2 | 30 | 10 | 32 | 47.2 | 20 | 6.23 | 5.66 | 9.12 | 2.48 | 2.32 | 6.34 | 4.45 | 4.54 | 1.95 |
| 59.2 | 25 | 15 | 32 | 37.2 | 30 | 6.01 | 6.15 | 2.30 | 2.42 | 2.64 | 9.26 | 4.87 | 5.08 | 4.31 |
| 69.2 | 15 | 15 | 65.33 | 3.87 | 30 | 7.05 | 7.13 | 1.08 | 2.91 | 3.03 | 4.17 | 5.19 | 5.63 | 8.54 |
| 74.2 | 20 | 5 | 82 | 7.2 | 10 | 7.10 | 6.64 | 6.48 | 2.74 | 2.58 | 5.82 | 5.03 | 4.82 | 4.11 |
| 64.2 | 30 | 5 | 48.67 | 40.53 | 10 | 5.56 | 5.67 | 1.85 | 2.14 | 2.20 | 2.43 | 3.85 | 4.27 | 10.91 |
| 5.42 | 40 | 5 | 15.33 | 73.87 | 10 | 4.76 | 4.69 | 1.49 | 1.92 | 1.81 | 6.00 | 4.14 | 3.72 | 10.23 |
| 49.2 | 45 | 5 | 2.4 | 86.8 | 10 | 4.71 | 4.31 | 8.54 | 1.52 | 1.66 | 9.04 | 3.210 | 3.50 | 9.10 |

The experimental values in Table 5 and Table 6 were obtained by experiments, glass samples prepared by fusing and cooling were ground and polished to a size of 20 mm×10 mm×1.5 mm for a spectrum test, an absorption spectrum was tested by a Perkin-ElmerL1 mbda900UV/VIS/NIR spectrophotometer, and a fluorescence spectrum was tested by a TRIAX320 fluorescence spectrometer (J-Y Company, France) under 808 pumping. A lifetime of the rare earth ions was obtained by a fluorescence intensity signal changed with time detected by an oscilloscope, and a lifetime of the fluorescence was a period of time that it took for a fluorescence intensity decayed to e⁻¹ of the highest intensity. All the tests were performed at a room temperature. Based on the tests, a calculation formula of the effective line width was:

$$Δ {λ}_{eff} = \int \frac{I ( λ ) d λ}{{I}_{max}} , \qquad ( 1 )$$

in the formula, Δλ_{eff} was the effective line width, Iₘₐₓ was a maximum light intensity in an emission spectrum, and I(λ)dλ was a product of the light intensity and a wavelength. The fluorescence full width at half maximum can be directly obtained from the emission spectrum. Based on the absorption spectrum, the absorption cross-section was calculated using a Beer-Lambert equation, and a calculation formula was:

$${σ}_{a} = \frac{2.3 \; 03 lg ( {I}_{0} / I )}{N l} , \qquad ( 2 )$$

wherein lg(I₀/I) was an absorption rate (also called an optical density) in the case of a certain light wavelength, N was a concentration of the rare earth ions in the glass, and l was a thickness of the glass. A calculation formula of the peak emission cross-section was:

$${σ}_{p} ( {λ}_{p} ) = \frac{{λ}_{p}^{4}}{8 π {cn}^{2} {λ}_{eff}} A , \qquad ( 3 )$$

wherein λₚ was a peak wavelength, c was a speed of light in vacuum (3×10⁸), n was a refractive index of the glass, Δλ_{eff} was the effective line width, A was a probability of radiative transition, and A is calculated by a Judd-Ofelt theory.

### Embodiment 5 Na₂O—MgO—P₂O₅ Ternary Glass System

Methods for predicting a density and a refractive index of the Na₂O—MgO—P₂O₅ ternary glass system were basically the same as those in Embodiment 3, except that the glass system was different. Predicted values of the glass systems have a relative error within 5% in comparison to the experimental values, which demonstrates that the methods for predicting the density and the refractive index of the ternary glass system are effective.

### Embodiment 6 TeO₂—BaO—Li₂O Ternary Glass System

Methods for predicting a density and a refractive index of the TeO₂—BaO—Li₂O ternary glass system were basically the same as those in Embodiment 3, except that the glass system was different. Predicted values of the glass systems have a relative error within 5% in comparison to the experimental values, which demonstrates that the methods for predicting the density and the refractive index of the ternary glass system are effective.

### Embodiment 7 SiO₂—B₂O₃—Al₂O₃Ternary Glass System

Methods for predicting a density and a refractive index of the SiO₂—B₂O₃—Al₂O₃ ternary glass system are basically the same as those in the Embodiment 3, except that the glass system is different. Predicted values of the glass systems have a relative error within 5% in comparison to the experimental values, which demonstrates that the methods for predicting the density and the refractive index of the ternary glass system are effective.

The methods for performance prediction of the binary and ternary glass systems provided by the present invention can be extended to a quaternary glass system, a quinary glass system and even a glass system with more components, such as a SiO₂—B₂O₃—CaO—Al₂O₃ glass system.

All the technical features of the above-described embodiments can be arbitrarily combined. In order to simplify the description, not all possible combinations of each of the technical features in the above-described embodiments are described. However, as long as there is no contradiction in the combinations of these technical features, these combinations should be considered as the scope recorded in this specification.

The above-described embodiments only express several implementations of the present invention, which are described more specifically and in details, but the embodiments cannot be understood as limiting the scope of protection of the present invention. It should be pointed out that several modifications and improvements can be made by those skilled in the art without deviating from the concept of the present invention, and all the modifications and improvements shall fall within the scope of protection of the present invention. Therefore, the scope of protection of the present invention shall be determined by the appended claims.

1. A method for performance prediction of a multiplex glass system comprising the following steps:
determining species of atoms for structural search according to components of the multiplex glass system;
performing structural search based on a first principle to search out compounds that can be formed by interaction between the atoms;
comparing a formation energy and a phonon spectrum of each of the compounds to obtain stable compounds;
constructing a glass structural composition diagram according to the stable compounds, microstructural units of glassy compounds adjacent to a composition point of a target glass are structural genes of the glass; and
calculating a property of the target glass according to a leverage model formula of the multiplex glass system, the leverage model formula of the multiplex glass system being P₀=Σᵢ₌₁ⁿPi×Li, wherein the multiplex glass system has n components, P₀ is the property of the target glass, Pi is a property of the structural gene of the target glass, and Li is a content of the structural gene of the target glass in the target glass.

2. A method for performance prediction of a binary glass system comprising the following steps:
performing structural search based on a first principle to search out compounds that can be formed between every two atoms or among every three atoms in components of a target glass, and obtaining formation energies and phonon spectrums of the compounds by calculating;
comparing the formation energies and the phonon spectrums of the compounds respectively to obtain stable compounds;
drawing a composition triangle by taking composition atoms of the target glass as vertexes, and marking coordinates of the stable compounds in the composition triangle to obtain a binary glass system composition diagram;
finding out a composition coordinate of the target glass in the binary glass system composition diagram, microstructural units of glassy compounds corresponding to two stable compounds adjacent to the composition coordinate are structural genes of the target glass; and
calculating a property of the target glass according to a leverage model formula of the binary glass system, the leverage model formula of the binary glass system being P₀=P1×L1+P2×L2, wherein P₀ is the property of the target glass, P1 and P2 are properties of the structural genes of the target glass, and L1 and L2 are contents of the structural genes of the target glass in the target glass.

3. The method for performance prediction of the binary glass system according to claim 2, wherein the property is at least one of a mechanical property, a magnetic property, an electrical property, a luminescent property and a thermal property.

4. The method for performance prediction of the binary glass system according to claim 2, wherein the property is at least one of a density, a refractive index, a fluorescence full width at half maximum, an effective line width, an absorption cross-section and a peak emission cross-section.

5. The method for performance prediction of the binary glass system according to claim 2, wherein performing the structural search based on the first principle is to perform high-throughput structural search using a first principle structural search software.

6. The method for performance prediction of the binary glass system according to claim 5, wherein a local particle swarm optimization algorithm is used in the high-throughput structural search, 35 to 50 structures are calculated for each iteration, and 20 to 30 iterations are calculated in total.

7. The method for performance prediction of the binary glass system according to claim 5, wherein the high-throughput structural search further comprises structure relaxation calculation, a cut-off energy of the structure relaxation is 400 ev to 500 ev, and a PBE functional in a generalized gradient approximation is used as a functional.

8. The method for performance prediction of the binary glass system according to claim 2, wherein before performing the structural search based on the first principle, the method further comprises determining a number range of each atom according to the species of the atoms in the components of the target glass.

9. The method for performance prediction of the binary glass system according to claim 2, wherein the step of comparing the formation energies and the phonon spectrums of the compounds respectively comprises:
constructing a bump map illustrating the formation energies of the compounds obtained by calculating which change with the components, and judging thermodynamically stable compounds in the compounds according to the bump map; and
calculating phonon spectrums of the thermodynamically stable compounds, and selecting a compound that does not contain an imaginary frequency in the phonon spectrum, which is namely the stable compound.

10. The method for performance prediction of the binary glass system according to claim 2, wherein the target glass comprises one or more of a laser glass, an optical glass, a biological glass, a nuclear technology glass, a safety glass and a ware glass.

11. A method for performance prediction of a ternary glass system comprising the following steps:
combining any two of three components of a target glass to obtain three binary composition systems, and performing structural search on each of the binary composition systems respectively according to the method for performance prediction of the binary glass system according to claim 2 to obtain corresponding stable compounds in each of the binary composition systems;
combining the three components of the target glass to obtain a ternary composition system, determining a proportion of four atoms in the ternary composition system, and performing structural search based on a first principle to search out compounds that can be formed by the four atoms in the ternary composition system;
comparing formation energies and phonon spectrums of the compounds that can be formed by the four atoms in the ternary composition system with formation energies and phonon spectrums of the stable compounds in the binary composition systems to determine stable compounds in the compounds that can be formed by the four atoms in the ternary composition system;
drawing a composition triangle by taking the components in the ternary composition system as vertexes, marking coordinates of all the stable compounds in the binary composition system and all the stable compounds in the ternary composition system in the composition triangle, taking the coordinates of all the stable compounds as vertexes, and dividing a triangular region according to a minimum area principle to obtain a ternary glass system composition diagram;
finding out a composition coordinate corresponding to the target glass in the ternary glass system composition diagram, microstructural units of glassy compounds corresponding to the compounds represented by three vertexes of the triangular region where the composition coordinate is located are structural genes of the target glass; and
calculating a property of the target glass according to a leverage model formula of the ternary glass system, the leverage model formula of the ternary glass system being P₀=P1×L1+P2×L2+P3×L3, wherein P₀ is the property of the target glass, P1, P2 and P3 are properties of the structural genes of the target glass, and L1, L2 and L3 are contents of the structural genes of the target glass in the target glass.

12. The method for performance prediction of the ternary glass system according to claim 11, wherein the step of comparing the formation energies and the phonon spectrums of the compounds that can be formed by the four atoms in the ternary composition system with the formation energies and the phonon spectrums of the stable compounds in the binary composition system comprises:
constructing a bump map illustrating the formation energies of the compounds that can be formed by the four atoms in the ternary composition system which change with the components by taking the stable compounds in the binary composition system as terminal vertexes of the components, and judging the thermodynamically stable compounds according to the bump map; and
calculating phonon spectrums of the thermodynamically stable compounds, and selecting a compound that does not contain an imaginary frequency in the phonon spectrum, which is namely the stable compound.

13. The method for performance prediction of the ternary glass system according to claim 11, wherein when no stable compound exists in the compounds that can be formed by the four atoms in the ternary composition system, all the stable compounds in the binary composition system are marked in the composition triangle only.

14. The method for performance prediction of the ternary glass system according to claim 11, wherein when the stable compound exists in the compounds that can be formed by the four atoms in the ternary composition system, all the stable compounds in the binary composition system and all the stable compounds in the ternary composition system are marked in the composition triangle.

15. (canceled)

16. (canceled)

17. (canceled)

18. (canceled)

19. (canceled)

20. (canceled)

21. The method for performance prediction of a ternary glass system according to claim 11, wherein the property is at least one of a mechanical property, a magnetic property, an electrical property, a luminescent property and a thermal property.

22. The method for performance prediction of a ternary glass system according to claim 11, wherein the property is at least one of a density, a refractive index, a fluorescence full width at half maximum, an effective line width, an absorption cross-section and a peak emission cross-section.

23. The method for performance prediction of a ternary glass system according to claim 11, wherein performing the structural search based on the first principle is to perform high-throughput structural search using a first principle structural search software.

24. The method for performance prediction of a ternary glass system according to claim 11, wherein a local particle swarm optimization algorithm is used in the high-throughput structural search, 35 to 50 structures are calculated for each iteration, and 20 to 30 iterations are calculated in total.

25. The method for performance prediction of a ternary glass system according to claim 11, wherein before performing the structural search based on the first principle, the method further comprises determining a number range of each atom according to the species of the atoms in the components of the target glass.

26. The method for performance prediction of a ternary glass system according to claim 11, wherein the target glass comprises one or more of a laser glass, an optical glass, a biological glass, a nuclear technology glass, a safety glass and a ware glass.
